(19) United States
(12) Patent Application Publication
(10) Pub. No.: US 2025/0304795 A1
(43) Pub. Date: Oct. 2, 2025

(54) Sulfonyl-bridged perinones

(71) Applicant: LANXESS Deutschland GmbH, Cologne (DE)

(72) Inventors: Sabine Schmauser, Leverkusen (DE); Marvin Kischkewitz, Düsseldorf (DE); Lars May, Langenfeld (DE)

(73) Assignee: LANXESS Deutschland GmbH, Cologne (DE)

(21) Appl. No.: 19/076,314

(22) Filed: Mar. 11, 2025

(30) Foreign Application Priority Data
Mar. 27, 2024 (EP) 24166725.2

(51) Int. Cl. C09B 57/12; C07D 519/00; C08K 5/41

(52) U.S. Cl. CPC C09B 57/12; C07D 519/00; C08K 5/41

(57) ABSTRACT

The invention relates to sulfonyl-bridged perinones in the form of compounds of the general formula (I) or isomeric forms thereof

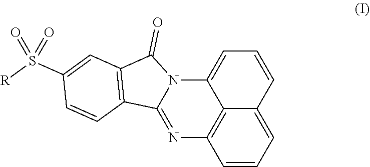

in which
R is C₁-C₆-alkyl, halogen, nitro, aryl, aryloxysulfonyl, hydroxy, C₁-C₆-alkoxy, aryloxy, optionally alkyl- or acyl-substituted amino, optionally alkyl- or aryl-substituted aminosulfonyl, or a fused-on cycloaliphatic or heterocyclic radical,
to a process for production thereof and to the use thereof for bulk colouring of plastics.

The invention relates to sulfonyl-bridged perinones and isomeric forms thereof, to a process for production thereof, and to the use thereof in plastic-based injection-moulded products, especially in injection-moulded products for the foods industry.

### BACKGROUND OF THE INVENTION

Plastics processible by injection moulding have also found wide use in the foods industry. For instance, EP 0 366 382 A1 teaches the use of polyamide for production of food and drink cans.

WO 2019/069292 A1 describes the use of polyethylene terephthalate (PET) for production of drinks bottles by blow moulding, especially injection blow moulding or stretch blow moulding. In principle, polyesters such as PET can alternatively be processed by injection moulding, extrusion or film blowing. In the form of Ultradur® B1520 FC R01 (FC=Food Contact), BASF SE offers a polybutylene terephthalate having high flowability that has been specially developed for the purpose of food packaging, which is notable for its stiffness, strength, and thermal and chemical stability.

However, plastic-based articles for food contact may be coloured with dyes either for aesthetic reasons or else for distinction of their contents. As of recently, however, dyes that have been in common use to date are no longer desirable in sensitive applications such as food contact, since they are suspected of constituting a risk to health; C. Barciela et al., Food and Chemical Toxicology Volume 178, August 2023, 113935, pages 1-12.

DE 43 27 855 A1 discloses, inter alia, optionally substituted benzenesulfonyl-bridged phthaloperinone dyes based on optionally substituted 1,8-diaminonaphthalene for bulk colouring of plastics. By dissolving these phthaloperinone dyes in monomeric components of a plastic and intimate mixing, transparent or muted bright orange to violet colours having good heat resistance and good light- and weatherfastness are obtained. DE 43 27 855 A1 does not make any statement as to the use of such phthaloperinone dyes in plastic-based injection-moulded products, especially those for the foods industry. The benzenesulfonyl-bridged phthaloperinone dyes according to DE 43 27 855 A1 are not red.

### SUMMARY OF THE INVENTION

It was therefore an object of the present invention to provide new red dyes that firstly have high thermal stability in order to be processed together with a plastic by the injection moulding method, but at the same time have low migration characteristics compared to dyes that are customarily usable in plastics for food contact.

The object is achieved by the subject-matter of the present invention in the form of sulfonyl-bridged perinones of the general formula (I) or isomers thereof

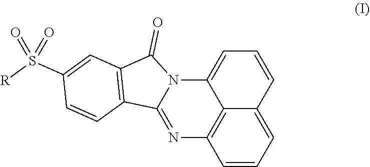

in which
R is C₁-C₆-alkyl, aryl, optionally alkyl- or acyl-substituted amino, optionally alkyl- or aryl-substituted aminosulfonyl, or a fused-on cycloaliphatic or heterocyclic radical.

In particular, the object is achieved by the subject-matter of the present invention in the form of optionally substituted 10,10′,9,9′ or 9,10 isomers of sulfonylbis-12H-phthaloperin-12-one.

Apart from their red colour, sulfonyl-bridged perinones of the formula (I) and isomeric forms thereof, especially optionally substituted 10,10′,9,9′ or 9,10 isomers of sulfonylbis-12H-phthaloperin-12-one, surprisingly have particularly high thermal stabilities, which makes them particularly suitable for use in injection moulding of plastics, and they are additionally notable for particularly low migration characteristics in or from the plastic compared to the dyes that are customarily used in the foods sector according to C. Barciela et al., Food and Chemical Toxicology Volume 178, August 2023, 113935, pages 1-12. Thermal stability is an important prerequisite for dyes that are used in plastics processible by injection moulding. In injection moulding, a polymer melt will be forced through the open die and the gate or gate system of an injection mould in the form of a shaping cavity under high pressure, usually in the range from 500 to 2000 bar, and at temperatures in the range from 200° C. to 300° C.; see:
https://www.kunststoffe.de/a/grundlagenartikel/spritzgiessen-254055.

For the sake of clarity, it should be noted that the scope of the present invention encompasses all the definitions and parameters cited below in general or specified in preferred ranges in any desired combinations. This likewise relates to the combination of stated amounts of individual components in relation to processes and uses claimed in accordance with the invention. Unless stated otherwise, the standards cited in the context of this application relate to the current version on the application date of the present invention. Unless stated otherwise, reported percentages are percentages by weight. The peri arrangement actually refers to the 1,8 arrangement in naphthalene. This meaning of the peri arrangement is applied both in the literature and in the context of the present application to arylene having more than two mutually fused benzene rings.

Aryl or an aryl group, abbreviated to Ar, is an organochemical radical having an aromatic base skeleton. Aryl is thus the general term for a single atom group deriving from aromatic hydrocarbons by removal of a hydrogen atom bonded to the ring. Most aryl radicals derive from benzene (C₆H₆); the simplest aryl group, which is preferred in accordance with the invention, is the phenyl group (Ph), (—C₆H₅).

Alkyl or an alkyl group is part of a molecule consisting of mutually bonded carbon and hydrogen atoms. The simplest alkyl group is the methyl group —CH₃. Further alkyl groups that are preferred in accordance with the invention are the ethyl group —CH₂—CH₃ or the n-propyl group —CH₂—CH₂—CH₃. The general formula for alkyl groups in the form of a chain is CₙH₂ₙ₊₁.

Acyl or an acyl group refers to a functional group having the general structure R—(C═O)— where R is an organyl radical, preferably alkyl, aryl or a heteroaromatic group, or a hydrogen atom. The acyl group is present in compounds such as aldehydes, or carboxylic acids, or carboxylic acid derivatives, especially carbonyl chlorides, in which, in a formal sense, an OH group, a hydrogen atom or a chlorine atom has been replaced by a radical.

By way of clarification, it should also be noted that the formula (I) reflects just one of several possible isomers.Formula (I) reflects just one of several possible isomers, where isomers, according to https://de.wikipedia.org./wiki/Isomerie, refer to the occurrence of two or more chemical compounds having the same empirical formula and molecular mass, but which differ in the linkage or spatial arrangement of the atoms and can be represented by different structural formulae. The inventive sulfonyl-bridged perinones of the formula (I) are positional isomers in which the same functional group can be localized at different positions, in the present case at position 9 or at position 10 of the perinone structure. By way of clarification, it should also be noted that the optionally substituted sulfonylbis-12H-phthaloperin-12-ones that are especially preferred in accordance with the invention take the form of their 10,10′,9,9′ or 9,10 isomers.

“Optionally substituted” or “unsubstituted” in the context of the present invention relates to the optionally substituted or unsubstituted naphthalene-1,8-diamine to be used in the process according to the invention, or the naphthyl radical, or to the two naphthyl radicals present therefrom in the inventive sulfonyl-bridged perinone of the formula (I) or in the especially preferred sulfonylbis-12H-phthaloperin-12-one. The two naphthyl radicals may preferably have one to a maximum of twelve identical or different substituents. In this case, the naphthalene-1,8-diamine to be used in the process according to the invention, in addition to the amino groups required for the reaction with 5,5-sulfonylbis(isobenzofuran-1,3-dione), has at least one further substituent. Preferably, for this purpose, at least one substituent should be selected from the group of chlorine, bromine, nitro, methoxy, NH₂, benzyloxy, hydroxy, —SO₂O(C₆H₅), —SO₂N(CH₃)₂, —SO₂NHCH₃, methyl, ethyl, n-propyl, isopropyl, n-, sec-, tert-butyl, NHCOCH₃, —N(C₂H₅)₂ and phenyl.

### PREFERRED EMBODIMENTS OF THE INVENTION

Isomers or isomeric forms of the 10,10′-sulfonyl-bridged perinones that are preferred in accordance with the invention are the 9,9′-sulfonyl-bridged perinones, the 10,9-sulfonyl-bridged perinones and the 9,10-sulfonyl-bridged perinones.

Inventive isomers or isomeric forms of the especially preferred optionally substituted sulfonylbis-12H-phthaloperin-12-ones are optionally substituted 10,10′-sulfonylbis-12H-phthaloperin-12-one, optionally substituted 9,10-sulfonylbis-12H-phthaloperin-12-one and optionally substituted 9,9-sulfonylbis-12H-phthaloperin-12-one.

The invention preferably relates to unsubstituted 10,10′-sulfonylbis-12H-phthaloperin-12-one of formula (II), unsubstituted 9,10-sulfonylbis-12H-phthaloperin-12-one of formula (III) and unsubstituted 9,9-sulfonylbis-12H-phthaloperin-12-one of formula (IV).

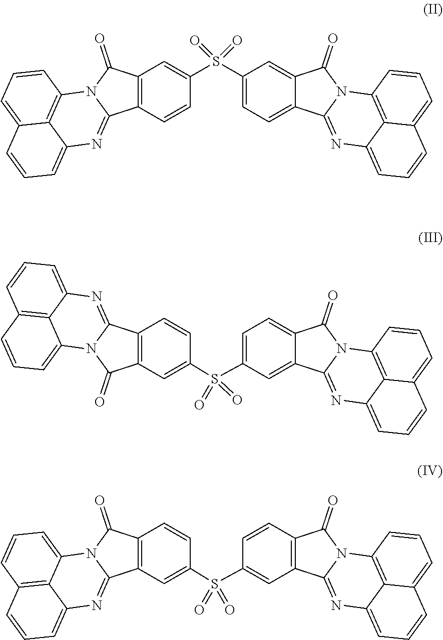 [SMILES: O=C1c2cc(S(=O)(=O)c3ccc4c(c3)C(=O)N3C4=Nc4cccc5cccc3c45)ccc2C2=Nc3cccc4cccc(c34)N12 ; O=C1c2cc(S(=O)(=O)c3ccc4c(c3)C3=Nc5cccc6cccc(c56)N3C4=O)ccc2C2=Nc3cccc4cccc(c34)N12 ; O=C1c2ccc(S(=O)(=O)c3ccc4c(c3)C3=Nc5cccc6cccc(c56)N3C4=O)cc2C2=Nc3cccc4cccc(c34)N12]

Substituted isomers of sulfonylbis-12H-phthaloperin-12-one of the formulae (II), (III) and (IV) that are especially preferred in accordance with the invention are notable for at least one substituent on at least one of the two naphthyl radicals. The two naphthyl radicals may preferably have one to a maximum of twelve identical or different substituents. Preferred substituents should be selected from the group of chlorine, bromine, nitro, methoxy, NH₂, benzyloxy, hydroxy, —SO₂O(C₆H₅), —SO₂N(CH₃)₂, —SO₂NHCH₃, methyl, ethyl, n-propyl, isopropyl, n-, sec-, tert-butyl, NHCOCH₃, —N(C₂H₅)₂ and phenyl.

Preferred plastics in the context of the present invention are those as used in injection moulding, extrusion or in blow moulding, especially in injection moulding. These are preferably thermoplastics, more preferably vinyl polymers, polyesters, polyolefins or polyamides. Preferred polyolefins are polyethylene or polypropylene. Preferred vinyl polymers are polystyrene, styrene-acrylonitrile copolymers, styrene-butadiene copolymers, styrene-butadiene-acrylonitrile terpolymers, polymethacrylate or polyvinylchloride. Preferred polyesters are polyethylene terephthalates, polybutylene terephthalates, polycarbonates or cellulose esters. Preferred polyamides are nylon-6 or nylon-6,6.

The invention preferably relates to sulfonyl-bridged perinones of the general formula (I) or isomers thereof

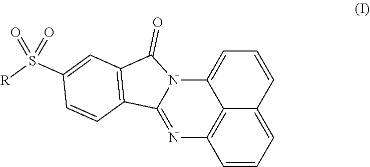

in which R is C₁-C₆-alkyl, aryl, optionally alkyl- or acyl-substituted amino, optionally alkyl- or aryl-substituted aminosulfonyl, or a fused-on cycloaliphatic or heterocyclic radical, having a colour distance ΔE<20 of the L*a*b* coordinates from a colour number beginning with “3” in the RAL colour chart.

The invention more preferably relates to sulfonyl-bridged perinones of the formula (I) or isomers thereof in which R is

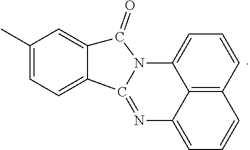 [SMILES: Cc1ccc2c(c1)C(=O)N1C2=Nc2cccc3cccc1c23]

In an alternative or preferred embodiment, the present invention relates to sulfonyl-bridged perinones of the formula (I) in which at least one of the phenyl rings in the perinone structure has at least one of the substituents from the group of chlorine, bromine, nitro, methoxy, NH₂, benzyloxy, hydroxy, —SO₂O(C₆H₅), —SO₂N(CH₃)₂, —SO₂NHCH₃, methyl, ethyl, n-propyl, isopropyl, n-, sec-, tert-butyl, NHCOCH₃, —N(C₂H₅)₂ or phenyl.

The sulfonyl group preferably bridges two of the same perinone structures. Very particularly preferably in accordance with the invention, the sulfonyl group bridges two 12H-phthaloperin-12-one radicals to one another, where 12H-phthaloperin-12-one is itself already known as Solvent Orange 60 dye [CAS No. 6925-69-5] from EP 1 245 645 A1. The synthesis thereof is described in EP 780 444 A2, example 1. The result of the inventive sulfonyl bridging of two 12H-phthaloperin-12-one radicals is the sulfonyl-bridged perinone dye of the formula (II) having the empirical formula C₃₆H₁₈N₄O₄S that is very particularly preferred in accordance with the invention, and 10,10′-sulfonylbis-12H-phthaloperin-12-one and isomers thereof.

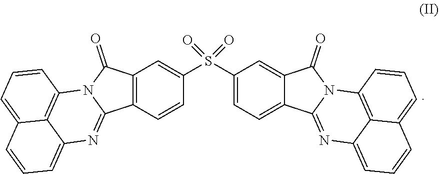 [SMILES: O=C1c2cc(S(=O)(=O)c3ccc4c(c3)C(=O)N3C4=Nc4cccc5cccc3c45)ccc2C2=Nc3cccc4cccc(c34)N12]

Isomers of 10,10′-sulfonylbis-12H-phthaloperin-12-one that are preferred in accordance with the invention are 9,9′-sulfonylbis-12H-phthaloperin-12-one, 10,9-sulfonylbis-12H-phthaloperin-12-one and 9,10-sulfonylbis-12H-phthaloperin-12-one.

TABLE 1
RAL colour chart
In the context of the present invention, red is considered to
mean a colour that, in the RAL colour system according to
https://de.wikipedia.org/wiki/RAL-Farbe#Rot, has a colour number
beginning with a “3” in the RAL colour chart.
More particularly, at the filing date of the present
invention, red hues are distinguished as follows:

| | | L* | a* | b* |
|---|---|---|---|---|
| RAL 3000 | Flame red | 44 | 50 | 39 |
| RAL 3001 | Signal red | 41 | 49 | 33 |
| RAL 3002 | Carmine red | 41 | 49 | 35 |
| RAL 3003 | Ruby red | 36 | 47 | 27 |
| RAL 3004 | Purple red | 31 | 38 | 18 |
| RAL 3005 | Wine red | 26 | 33 | 15 |
| RAL 3007 | Black red | 23 | 17 | 7 |
| RAL 3009 | Oxide red | 29.27 | 24.59 | 16.51 |
| RAL 3011 | Brown red | 34.52 | 28.66 | 13.44 |
| RAL 3012 | Beige red | 63.81 | 20.79 | 20.45 |
| RAL 3013 | Tomato red | 40.70 | 36.67 | 21.37 |
| RAL 3014 | Antique pink | 60.17 | 32.49 | 12.58 |
| RAL 3015 | Light pink | 71.23 | 21.59 | 4.98 |
| RAL 3016 | Coral red | 44.70 | 37.92 | 23.96 |
| RAL 3017 | Rose | 54.24 | 44.26 | 16.87 |
| RAL 3018 | Strawberry red | 50.77 | 49.15 | 19.86 |
| RAL 3020 | Traffic red | 46 | 59 | 54 |
| RAL 3022 | Salmon pink | 56.06 | 38.90 | 29.70 |
| RAL 3024 | Luminous red | 51.32 | 82.52 | 71.62 |
| RAL 3026 | Luminous bright red | 59 | 70 | 59 |
| RAL 3027 | Raspberry red | 43.07 | 46.96 | 15.81 |
| RAL 3028 | Pure red | 51 | 58 | 46 |
| RAL 3031 | Orient red | 46 | 45 | 25 |
| RAL 3032 | Pearl ruby red | 26.88 | 41.34 | 19.40 |
| RAL 3033 | Peal pink | 44.29 | 45.11 | 28.62 |

The table shows the apparatus-independent CIE L*a*b* colour values for the respective RAL values for red: L* stands for luminance, a* describes the colour locus with respect to the red-green axis and b* describes the colour locus with respect to the yellow-blue axis using D65 standard light with a 10° field of view of a standard observer. The colour model is standardized in EN ISO 11664-4 “Colorimetry-Part 4: CIE 1976 L*a*b* Colour space”. For L*a*b* colour space (also: CIELAB), see: https://de.wikipedia.org/wiki/Lab-Farbraum. Each colour in the colour space is defined by a colour locus having the Cartesian coordinates {L*, a*, b*}. The a*b* coordinate plane was constructed using opponent colour theory. Green and red are at opposite ends of the a* axis from one another and the b* axis runs from blue to yellow. Complementary hues are respectively opposite one another at a 180° angle; all achromatic colours are arranged in the middle thereof (the coordinate origin a*=0, b*=0).

The L* axis describes the brightness (luminance) of the colour with values of 0 to 100. In the diagram, it stands perpendicular to the a*b* plane at the origin. It may also be referred to as the neutral grey axis since all achromatic colours (grey hues) are contained between the endpoints of black (L*=0) and white (L*=100). The a* axis describes the green or red component of a colour, where negative values represent green and positive values represent red. The b* axis describes the blue or yellow component of a colour, where negative values represent blue and positive values represent yellow.

The a* values range from approximately −170 to +100 and the b* values from −100 to +150, the maximum values being achieved only at moderate brightness of certain hues. The CIELAB colour entity has its greatest extent in the region of moderate brightness, but this differs in height and size depending on the colour range.

According to the invention, hues similar to red that have a colour distance ΔE<20 of the L*a*b* coordinates from a colour number beginning with “3” in the RAL colour chart for the colour red are also included, however.

Preference is given in accordance with the invention to red sulfonyl-bridged perinones of the general formula (I) or isomers thereof that have a colour distance ΔE<20 of the L*a*b* coordinates from a colour number beginning with “3” in the RAL colour chart for the colour red, with a yellow tinge where a*>0 and b*≥25. The following is thus applicable to the hue angle h: h°ₘᵢₙᵢₘᵤₘ=14° and h°ₘₐₓᵢₘᵤₘ<90°, with inclusion of all colour loci in between.

Especially preferred are optionally substituted 10,10′,9,9′ or 9,10 isomers of sulfonylbis-12H-phthaloperin-12-one having a colour distance ΔE<20 of the L*a*b* coordinates from a colour number beginning with “3” in the RAL colour chart according to the colour model in EN ISO 11664-4. The colour numbers beginning with “3” in the RAL colour chart according to the colour model in EN ISO 11664-4 represent the colour red.

Especially preferred in particular are optionally substituted 10,10′,9,9′ or 9,10 isomers of sulfonylbis-12H-phthaloperin-12-one having a colour distance ΔE<20 of the L*a*b* coordinates from a colour number beginning with “3” in the RAL colour chart according to the colour model in EN ISO 11664-4, with a yellow tinge defined as a*>0 and b*≥25, which means that the following is applicable to the hue angle h: h°ₘᵢₙᵢₘᵤₘ=14° and h°ₘₐₓᵢₘᵤₘ<90°, with inclusion of all colour loci in between.

Also especially preferred are unsubstituted 10,10′,9,9′ or 9,10 isomers of sulfonylbis-12H-phthaloperin-12-one having a colour distance ΔE<20 of the L*a*b* coordinates from a colour number beginning with “3” in the RAL colour chart according to the colour model in EN ISO 11664-4. The colour numbers beginning with “3” in the RAL colour chart according to the colour model in EN ISO 11664-4 represent the colour red.

Especially preferred in particular, finally, are unsubstituted 10,10′,9,9′ or 9,10 isomers of sulfonylbis-12H-phthaloperin-12-one having a colour distance ΔE<20 of the L*a*b* coordinates from a colour number beginning with “3” in the RAL colour chart according to the colour model in EN ISO 11664-4, with a yellow tinge defined with a*>0 and b*≥25, which means that the following is applicable to the hue angle h: h°ₘᵢₙᵢₘᵤₘ=14° and h°ₘₐₓᵢₘᵤₘ<90°, with inclusion of all colour loci in between.

### Process

One possible synthesis route for preparation of sulfonyl-bridged perinones of the formula (I) and isomeric forms thereof is the reaction or condensation of sulfonyl-substituted isobenzofurandiones with at least one aromatic diamine from the group of naphthylene-1,8-diamine, chloronaphthylene-1,8-diamine, dichloronaphthylene-1,8-diamine, methylnaphthylene-1,8-diamine, dimethylnaphthylene-1,8-diamine, methoxynaphthylene-1,8-diamine, ethoxynaphthylene-1,8-diamine, acetamino-1,8-naphthylenediamine and 1,8-diaminoacenaphthylene. Particular preference is given to naphthylene-1,8-diamine. This synthesis route should be employed with particular preference for preparation of the unsubstituted sulfonyl-bridged perinone of the formula (II) and isomeric forms thereof that are especially preferred in accordance with the invention, for which 5,5-sulfonylbis(isobenzofuran-1,3-dione) is reacted with naphthalene-1,8-diamine.

Preference is given to using at least 2 mol of optionally substituted aromatic diamine per mole of 5,5-sulfonyl-bridged isobenzofurandione. In some embodiments, it is also possible to use more than 2 mol of the optionally substituted aromatic diamine, although preference is given to stoichiometric use of the reactants.

Since at least 2 mol of optionally substituted aromatic diamine is used per mole of sulfonyl-substituted isobenzofurandione for synthesis of inventive sulfonylbis-12H-phthaloperin-12-ones, it is also possible to prepare sulfonylbis-12H-phthaloperin-12-ones in which the two 1,8-naphthyl radicals have different substitution. What is meant by “different substitution” in this case is substitution at different positions on the respective naphthyl radical and/or different substituents on the respective naphthyl radical from the group of chlorine, bromine, nitro, methoxy, NH₂, benzyloxy, hydroxy, —SO₂O(C₆H₅), —SO₂N(CH₃)₂, —SO₂NHCH₃, methyl, ethyl, n-propyl, isopropyl, n-, sec-, tert-butyl, NHCOCH₃, —N(C₂H₅)₂ and phenyl. The reactants can be condensed directly by melting appropriate molar equivalents of the reactants together at a temperature in the range from 120° C. to 250° C.

Preference is given to converting the reactants in a solvent at a temperature in the range from 110° C. to 220° C., optionally under pressure, where the water of reaction can be removed by distillation. For the condensation, preference is given to using at least one solvent selected from the group of chlorobenzene, o-dichlorobenzene, trichlorobenzene, xylene, dimethylformamide, N-methylpyrrolidone, glacial acetic acid, propionic acid, phenol, cresols, phenoxyethanol, glycols and the mono- and dialkyl ethers thereof, alcohols, especially methanol, ethanol, i-propanol, water and aqueous solvents, especially dilute sulfuric acid. Particular preference is given in accordance with the invention to using phenol.

In one embodiment, the reaction can be effected with addition of at least one acidic catalyst. Preference is given in this case to using catalysts selected from the group of zinc chloride, p-toluenesulfonic acid, hydrochloric acid, sulfuric acid, organic acids.

A reaction mixture for preparation of the sulfonyl-bridged perinones of the formula (I) and isomeric forms thereof, especially for preparation of optionally substituted sulfonylbis-12H-phthaloperin-12-ones in the form of their 10,10′,9,9′ or 9,10 isomers, is preferably worked up by diluting with alcohols, preferably methanol, ethanol, propanol or butanol. It is also possible to use aromatic diluents, preferably chlorobenzene or toluene. The process according to the invention affords the sulfonyl-bridged perinones of the formula (I) and isomeric forms thereof preferably in yields of 85% to 95% of theory.

The inventive sulfonyl-bridged perinones of the formula (I) and isomeric forms thereof, especially the optionally substituted sulfonylbis-12H-phthaloperin-12-ones in the form of their 10,10′,9,9′ or 9,10 isomers, are of excellent suitability for bulk colouring of plastics. What is meant here more particularly by bulk colouring is methods in which the dye is incorporated into the molten plastic mass, preferably with the aid of an extruder, or in which the dye is added directly to reactants for production of a particular plastic, preferably the respective monomers prior to polymerization.

Particularly preferred plastics to be coloured by means of inventive sulfonyl-bridged perinones of the formula (I) and isomeric forms thereof, especially by means of optionally substituted sulfonylbis-12H-phthaloperin-12-ones in the form of their 10,10′,9,9′ or 9,10 isomers, are thermoplastics usable in injection moulding, preferably vinyl polymers, polyesters, polyolefins or polyamides, especially plastics for injection moulding. Preferred polyolefins are polyethylene or polypropylene. Preferred vinyl polymers are polystyrene, styrene-acrylonitrile copolymers, styrene-butadiene copolymers, styrene-butadiene-acrylonitrile terpolymers, polymethacrylate or polyvinylchloride. Preferred polyesters are polyethylene terephthalates, polybutylene terephthalates, polycarbonates or cellulose esters. Preferred polyamides are nylon-6 or nylon-6,6.

Very particularly preferred plastics to be coloured by means of inventive sulfonyl-bridged perinones of the formula (I) and appropriate isomers, especially by means of optionally substituted sulfonylbis-12H-phthaloperin-12-ones in the form of their 10,10′,9,9′ or 9,10 isomers, are polystyrene, styrene-acrylonitrile copolymers, styrene-butadiene copolymers, styrene-butadiene-acrylonitrile terpolymers, polymethacrylate, polyethylene terephthalate, nylon-6 or nylon-6,6.

The sulfonyl-bridged perinones of the formula (I) and appropriate isomers that are to be used as dyes in accordance with the invention, especially the optionally substituted sulfonylbis-12H-phthaloperin-12-ones in the form of their 10,10′,9,9′ or 9,10 isomers, are preferably employed in finely divided form, optionally with additional use of at least one dispersant.

If the sulfonyl-bridged perinones of the formula (I) and appropriate isomers that are to be used as dyes in accordance with the invention, especially the optionally substituted sulfonylbis-12H-phthaloperin-12-ones in the form of their 10,10′,9,9′ or 9,10 isomers, are used after polymerization of the respective plastic, they are mixed or ground in dry form with the pelletized plastic, and this mixture is preferably plastified and homogenized on mixing rolls or in screw systems. It is alternatively possible to add the sulfonyl-bridged perinones of the formula (I) and isomeric forms thereof that are to be used as dyes in accordance with the invention, especially the optionally substituted sulfonylbis-12H-phthaloperin-12-ones in the form of their 10,10′,9,9′ or 9,10 isomers, to the molten mass of a plastic and to distribute them homogeneously therein by stirring. Such a precoloured plastic is then typically or preferably processed further by spinning to give bristles or filaments, or by extrusion, blow moulding or injection moulding to give shaped articles.

Since the sulfonyl-bridged perinones of the formula (I) and appropriate isomers that are to be used as dyes in accordance with the invention, especially the optionally substituted sulfonylbis-12H-phthaloperin-12-ones in the form of their 10,10′,9,9′ or 9,10 isomers, are stable toward polymerization catalysts, especially toward peroxides, it is also possible to add these to the monomeric reactants of the respective plastics and then to polymerize the coloured monomeric reactants in the presence of polymerization catalysts. For this purpose, the inventive sulfonyl-bridged perinones of the formula (I) and isomeric forms thereof, especially the optionally substituted sulfonylbis-12H-phthaloperin-12-ones in the form of their 10,10′,9,9′ or 9,10 isomers, are preferably dissolved in or mixed intimately with the monomeric reactants of a particular plastic. Preferably, the inventive sulfonyl-bridged perinones of the formula (I) and isomeric forms thereof, especially the optionally substituted sulfonylbis-12H-phthaloperin-12-ones in the form of their 10,10′,9,9′ or 9,10 isomers, are used for colouring of the plastics or polymers mentioned in amounts in the range from 0.0001% to 1% by weight, especially in the range from 0.01% to 0.5% by weight, based in each case on the amount of plastic or polymer.

By adding pigments that are insoluble in the particular plastic or in the polymers, especially titanium dioxide, it is possible to obtain corresponding valuable muted colours. These insoluble pigments, especially titanium dioxide, are preferably used in amounts in the range from 0.01% to 10% by weight, preferably in the range from 0.1% to 5% by weight, based on the amount of polymer.

According to the invention, it is also possible to use mixtures of different inventive sulfonyl-bridged perinones and/or mixtures of inventive sulfonyl-bridged perinones of the formula (I) and isomeric forms thereof, especially mixtures of the optionally substituted sulfonylbis-12H-phthaloperin-12-ones in the form of their 10,10′,9,9′ or 9,10 isomers, with other dyes and/or inorganic or organic pigments.

### Preferred Methods of Use

The present invention relates to the use of sulfonyl-bridged perinones of the formula (I) and corresponding isomers, especially to the use of optionally substituted sulfonylbis-12H-phthaloperin-12-ones in the form of their 10,10′,9,9′ or 9,10 isomers, for bulk colouring of plastics with simultaneous reduction in migration characteristics in said plastics, determinable according to DIN 53775-3.

The plastics are preferably those that are used in injection moulding, in extrusion or in blow moulding. The plastics are preferably vinyl polymers, polyesters, polyolefins or polyamides, especially preferably nylon-6 or polycarbonate.

They are preferably plastics for injection moulding. Preferred polyolefins are polyethylene or polypropylene. Preferred vinyl polymers are polystyrene, styrene-acrylonitrile copolymers, styrene-butadiene copolymers, styrene-butadiene-acrylonitrile terpolymers, polymethacrylate or polyvinylchloride. Preferred polyesters are polyethylene terephthalates, polybutylene terephthalates, polycarbonates or cellulose esters. Preferred polyamides are nylon-6 or nylon-6,6.

The use preferably relates to sulfonyl-bridged perinones of the formula (I) and isomers thereof, in which R is a radical

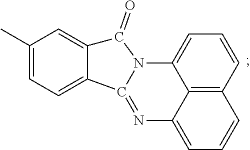 [SMILES: Cc1ccc2c(c1)C(=O)N1C2=Nc2cccc3cccc1c23]

particular preference is given to the use of 10,10′-sulfonylbis-12H-phthaloperin-12-one and isomers thereof. Especially preferably, in this case, all naphthyl radicals are unsubstituted. The use therefore preferably relates to the unsubstituted 10,10′,9,9′ or 9,10 isomers of sulfonylbis-12H-phthaloperin-12-one. The present invention alternatively relates to the use of sulfonyl-bridged perinones of the formula (I) and corresponding isomers, especially to the use of optionally substituted sulfonylbis-12H-phthaloperin-12-ones in the form of their 10,10′,9,9′ or 9,10 isomers, as dyes for bulk colouring of plastics with a simultaneous increase in the thermal stability of polymer compositions based on these dyes in these plastics, determinable according to DIN EN 12877. The plastics are preferably those for injection moulding, blow moulding or extrusion, especially for injection moulding. Preferred plastics are vinyl polymers, polyesters, polyolefins or polyamides, especially preferably nylon-6 or polycarbonate. Preferred polyolefins are polyethylene or polypropylene. Preferred vinyl polymers are polystyrene, styrene-acrylonitrile copolymers, styrene-butadiene copolymers, styrene-butadiene-acrylonitrile terpolymers, polymethacrylate or polyvinylchloride. Preferred polyesters are polyethylene terephthalates, polybutylene terephthalates, polycarbonates or cellulose esters. Preferred polyamides are nylon-6 or nylon-6,6.

The use preferably relates to sulfonyl-bridged perinones of the formula (I) and isomers thereof, in which R is a radical

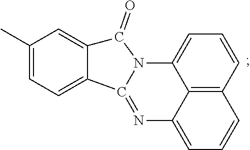 [SMILES: Cc1ccc2c(c1)C(=O)N1C2=Nc2cccc3cccc1c23]

particular preference is given to 10,10′-sulfonylbis-12H-phthaloperin-12-one and isomers thereof.

The invention preferably relates to the use of optionally substituted 10,10′,9,9′ or 9,10 isomers of sulfonylbis-12H-phthaloperin-12-one for bulk colouring of plastics in order to achieve a colour distance ΔE<20 of the L*a*b* coordinates from a colour number beginning with “3” in the RAL colour chart according to the colour model in EN ISO 11664-4. The colour numbers beginning with “3” in the RAL colour chart represent the colour red.

Preference is given to the use of optionally substituted 10,10′,9,9′ or 9,10 isomers of sulfonylbis-12H-phthaloperin-12-one for bulk colouring of plastics in order to achieve a colour distance ΔE<20 of the L*a*b* coordinates from a colour number beginning with “3” in the RAL colour chart according to the colour model in EN ISO 11664-4, with a yellow tinge defined with a*>0 and b*≥25, which means that the following is applicable to the hue angle h: h°ₘᵢₙᵢₘᵤₘ=14° and h°ₘₐₓᵢₘᵤₘ<90°, with inclusion of all colour loci in between.

Especially preferred is the use of optionally substituted 10,10′,9,9′ or 9,10 isomers of sulfonylbis-12H-phthaloperin-12-one for bulk colouring of plastics in order to achieve a colour distance ΔE<20 of the L*a*b* coordinates from a colour number beginning with “3” in the RAL colour chart according to the colour model in EN ISO 11664-4, with a simultaneous increase in the thermal stability of polymer compositions based on these isomers of sulfonylbis-12H-phthaloperin-12-one in plastics, determinable according to DIN EN 12877, where the increase in thermal stability observed in the context of the present invention for the plastics examined was within the scope of the processing limits thereof in the polymer compositions examined.

The plastics here too are preferably those for injection moulding, blow moulding or extrusion, especially for injection moulding. The plastics here too are preferably vinyl polymers, polyesters, polyolefins or polyamides, especially preferably nylon-6 or polycarbonate. Preferred polyolefins are polyethylene or polypropylene. Preferred vinyl polymers here too are polystyrene, styrene-acrylonitrile copolymers, styrene-butadiene copolymers, styrene-butadiene-acrylonitrile terpolymers, polymethacrylate or polyvinylchloride. Preferred polyesters are polyethylene terephthalates, polybutylene terephthalates, polycarbonates or cellulose esters. Preferred polyamides here too are nylon-6 or nylon-6,6. This use preferably also relates to unsubstituted 10,10′-sulfonylbis-12H-phthaloperin-12-one and the 9,9′ and 9,10 isomers thereof.

### Preferred Products

The present invention finally also relates to extruded, blow-moulded or injection-moulded products based on at least one sulfonyl-bridged perinone of the general formula (I) or isomeric forms thereof

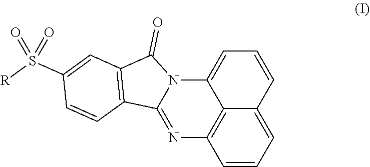

in which
R is C₁-C₆-alkyl, aryl, optionally alkyl- or acyl-substituted amino, optionally alkyl- or aryl-substituted aminosulfonyl, or a fused-on cycloaliphatic or heterocyclic radical, and
at least one plastic, preferably at least one thermoplastic, more preferably at least one plastic from the group of vinyl polymers, polyesters, polyolefins and polyamides. Preferred polyolefins are polyethylene or polypropylene. Preferred vinyl polymers are polystyrene, styrene-acrylonitrile copolymers, styrene-butadiene copolymers, styrene-butadiene-acrylonitrile terpolymers, polymethacrylate or polyvinylchloride. Preferred polyesters are polyethylene terephthalates, polybutylene terephthalates, polycarbonates or cellulose esters. Preferred polyamides are nylon-6 or nylon-6,6.

Preference is given to those extruded, blow-moulded or injection-moulded products that are based on sulfonyl-bridged perinones of the general formula (I) or isomers thereof, in which R is a Very particular preference is given to extruded, blow-moulded or injection-moulded products that are based on 10,10′-sulfonylbis-12H-phthaloperin-12-one and isomers thereof.

The present invention relates more particularly to extruded, blow-moulded or injection-moulded products, preferably extruded, blow-moulded or injection-moulded products for food contact, that are based on at least one optionally substituted sulfonylbis-12H-phthaloperin-12-one in the form of its 10,10′,9,9′ or 9,10 isomers and at least one plastic, preferably at least one thermoplastic, more preferably at least one plastic from the group of vinyl polymers, polyesters, polyolefins and polyamides. Preferred polyolefins are polyethylene or polypropylene. Preferred vinyl polymers are polystyrene, styrene-acrylonitrile copolymers, styrene-butadiene copolymers, styrene-butadiene-acrylonitrile terpolymers, polymethacrylate or polyvinylchloride. Preferred polyesters are polyethylene terephthalates, polybutylene terephthalates, polycarbonates or cellulose esters. Preferred polyamides are nylon-6 or nylon-6,6.

Preference is given to those extruded, blow-moulded or injection-moulded products, preferably extruded, blow-moulded or injection-moulded products for food contact, that are based on unsubstituted 10,10′,9,9′ or 9,10 isomers of sulfonylbis-12H-phthaloperin-12-one.

Especially preferred in particular are those extruded, blow-moulded or injection-moulded products, preferably extruded, blow-moulded or injection-moulded products for food contact, that are based on unsubstituted 10,10′,9,9′ or 9,10 isomers of sulfonylbis-12H-phthaloperin-12-one and have a colour distance ΔE<20 of the L*a*b* coordinates from a colour number beginning with “3” in the RAL colour chart according to the colour model in EN ISO 11664-4. Very particular preference is given to those extruded, blow-moulded or injection-moulded products that additionally have a yellow tinge defined with a*>0 and b*≥25, which means that the following is applicable to the hue angle h: h°ₘᵢₙᵢₘᵤₘ=14° and h°ₘₐₓᵢₘᵤₘ<90°, with inclusion of all colour loci in between.

It will be understood that the specification and examples are illustrative but not limitative of the present invention and that other embodiments within the spirit and scope of the invention will suggest themselves to those skilled in the art.

### EXAMPLES

### Example 1

63 g of phenol was melted at a temperature of 80° C. To this was added 5.27 g (32.7 mmol) of 1,8-diaminonaphthalene, and the mixture was stirred for 30 minutes. Subsequently, 6.32 g (16.8 mmol) of 5,5-sulfonylbis(isobenzofuran-1,3-dione) was introduced. This reaction mixture was heated to 135° C. and kept at that temperature for 6 hours, and the resultant water of reaction was distilled off. The reaction mixture was then cooled down to 100° C. within 1.5 h, and 65 g of methanol was added. The reaction mixture was stirred for a further 10 hours and cooled down to 30° C. The reaction product was isolated using a suction filter and washed first with 40 g of methanol and then washed with 400 g of water. After drying in a vacuum drying cabinet at 80° C. and 150 mbar, an isomer mixture comprising essentially 10,10′-sulfonylbis-12H-phthaloperin-12-one of formula (II) was obtained in a yield of 99% with red colour close to RAL 3028.

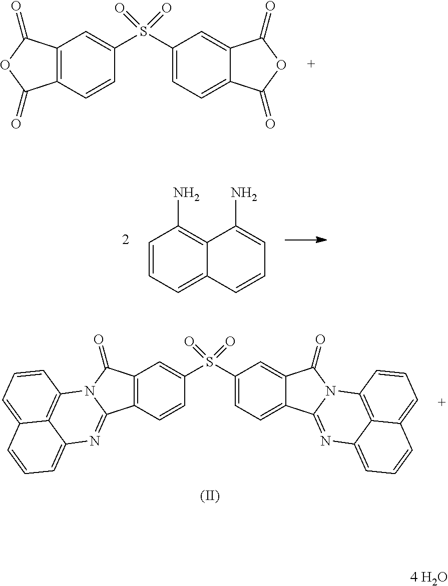 [SMILES: O=C1OC(=O)c2cc(S(=O)(=O)c3ccc4c(c3)C(=O)OC4=O)ccc21 ; Nc1cccc2cccc(N)c12 ; O=C1c2cc(S(=O)(=O)c3ccc4c(c3)C(=O)N3C4=Nc4cccc5cccc3c45)ccc2C2=Nc3cccc4cccc(c34)N12 ; "(II)"]

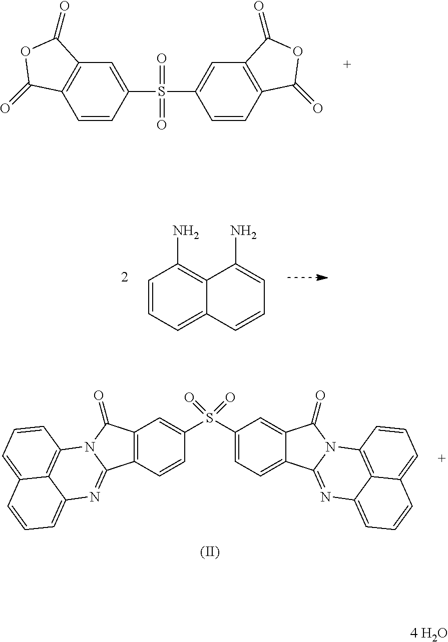 [SMILES: O=C1OC(=O)c2cc(S(=O)(=O)c3ccc4c(c3)C(=O)OC4=O)ccc21 ; Nc1cccc2cccc(N)c12 ; O=C1c2cc(S(=O)(=O)c3ccc4c(c3)C(=O)N3C4=Nc4cccc5cccc3c45)ccc2C2=Nc3cccc4cccc(c34)N12 ; "(II)"]

The formula (II) of the sulfonyl-bridged 10,10′-sulfonylbis-12H-phthaloperin-12-one dye as shown in the reaction scheme represents only one form of possible isomers that can arise in the synthesis.

By way of evidence of the improvements in the properties described in the description, corresponding polymer compositions were first produced by compounding from polycarbonate (polymer composition 1) and from nylon-6 (polymer composition 2) with 10,10′-sulfonylbis-12H-phthaloperin-12-one and its isomers. To this end, the individual components were mixed in a twin-screw extruder (Leistritz LSM 30-34 from Leistritz AG (Nuremberg, Germany)) at temperatures between 240° C. and 260° C., extruded, cooled until pelletizable and pelletized. After drying (generally for two days at 80° C. in a vacuum drying cabinet), the pellets were processed by injection moulding at temperatures in the range from 240 to 300° C. to give standard test specimens for the respective tests.

In the context of the present invention, migration of the 10,10′-sulfonylbis-12H-phthaloperin-12-one and its isomers was examined using plaques of dimensions 60·40·2 mm³ based on the thermoplastics polycarbonate and nylon-6 according to standard DIN 53775-3. This was done by clamping the test specimen dyed with the sulfonyl-bridged perinone having the empirical formula C₃₆H₁₈N₄O₄S between two uncoloured test specimens having a pressure of 4 N/cm² at a temperature of 80° C. for 24 h and then visually assessing migration based on dye residues on the test specimens that were initially colourless.

Likewise in the context of the present invention, the thermal stability of the moulding compounds described in Tab. I that had been coloured with 10,10′-sulfonylbis-12H-phthaloperin-12-one and its isomers was assessed by means of the 60·40·2 mm³ plaques, in accordance with standard DIN EN 12877.

Component A): polycarbonate (Makrolon® 2800, from Covestro AG, Germany)

Component B): nylon-6 (Durethan® B30S, from Lanxess Deutschland GmbH, Cologne, Germany)

Component C): isomers of 10,10′-sulfonylbis-12H-phthaloperin-12-one according to Example 1

TABLE 2

| | | Polymer | Polymer |
|---|---|---|---|
| | | composition 1 | composition 2 |
| Component A) | parts by mass | 99.8 | — |
| Component B) | parts by mass | — | 99.8 |
| Component C) | parts by mass | 0.2 | 0.2 |
| Migration | | none apparent | none apparent |
| Thermal stability | ° C. | 350 | 280 |

The results in Tab. 2, for 10,10′-sulfonylbis-12H-phthaloperin-12-one and its isomers, do not show any apparent migration thereof into or out of polymer compositions 1 and 2, and the polymer plaques based on 10,10′-sulfonylbis-12H-phthaloperin-12-one and its isomers, and also polycarbonate and nylon-6, are notable for very high thermal stability that extended up to the processing limits of the polymer compositions examined.

It will be understood that the specification and examples are illustrative but not limitative of the present invention and that other embodiments within the spirit and scope of the invention will suggest themselves to those skilled in the art.

1. A sulfonyl-bridged perinone of the general formula (I) or isomeric forms thereof 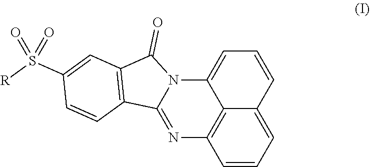
wherein
R is C₁-C₆-alkyl, aryl, optionally alkyl- or acyl-substituted amino, optionally alkyl- or aryl-substituted aminosulfonyl, or a fused-on cycloaliphatic or heterocyclic radical.

2. The sulfonyl-bridged perinone according to claim 1, wherein R is a radical

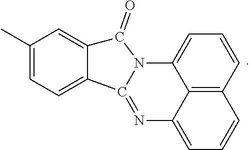 [SMILES: Cc1ccc2c(c1)C(=O)N1C2=Nc2cccc3cccc1c23]

3. The sulfonyl-bridged perinone according to claim 1, wherein at least one of the phenyl rings has at least one of the substituents selected from the group consisting of chlorine, bromine, nitro, methoxy, NH₂, benzyloxy, hydroxy, —SO₂O(C₆H₅), —SO₂N(CH₃)₂, —SO₂NHCH₃, methyl, ethyl, n-propyl, isopropyl, n-, sec-, tert-butyl, NHCOCH₃, —N(C₂H₅)₂ and phenyl.

4. The sulfonyl-bridged perinone according to claim 1, wherein formula (I) is 10,10′-sulfonylbis-12H-phthaloperin-12-one.

5. The sulfonyl-bridged perinone according to claim 1, having a colour distance ΔE<20 of the L*a*b* coordinates from a colour number beginning with “3” in the RAL colour chart for the colour red.

6. The sulfonyl-bridged perinone according to claim 5, having a yellow tinge where a*>0 and b*≥25.

7. A method of bulk colouring plastic comprising dissolving a sulfonyl-bridged perinone of the formula (I) or isomeric forms thereof 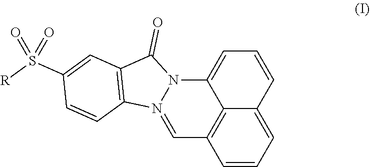
wherein
R is C₁-C₆-alkyl, halogen, nitro, aryl, aryloxysulfonyl, hydroxy, C₁-C₆-alkoxy, aryloxy, optionally alkyl- or acyl-substituted amino, optionally alkyl- or aryl-substituted aminosulfonyl, or a fused-on cycloaliphatic or heterocyclic radical in the plastic.

8. The method according to claim 7, wherein at least one of the phenyl rings in formula (I) has at least one of the substituents from the group consisting of chlorine, bromine, nitro, methoxy, NH₂, benzyloxy, hydroxy, —SO₂O(C₆H₅), —SO₂N(CH₃)₂, —SO₂NHCH₃, methyl, ethyl, n-propyl, isopropyl, n-, sec-, tert-butyl, NHCOCH₃, —N(C₂H₅)₂ and phenyl.

9. The method according to claim 7, wherein in formula (I) the sulfonyl bridge is disubstituted by the same radicals.

10. The method according to claim 9, wherein in formula (I) the sulfonyl bridge bridges the same two 12H-phthaloperin-12-one radicals to one another.

11. The method according to claim 10, wherein formula (I) is 10,10′-sulfonylbis-12H-phthaloperin-12-one.

12. The method according to claim 7, wherein the plastic is a thermoplastic for extrusion, a thermoplastic for blow moulding or a thermoplastic for injection moulding.

13. The method according to claim 12, wherein the plastic is a vinyl polymer, a polyester, a polyolefin or a polyamide.

14. The method according to claim 13, wherein the polyolefin is selected from polyethylene or polypropylene, the vinyl polymer is selected from polystyrene, a styrene-acrylonitrile copolymer, a styrene-butadiene copolymer, a styrene-butadiene-acrylonitrile terpolymer, polymethacrylate or polyvinylchloride, the polyester is selected from polyethylene terephthalate, polybutylene terephthalate, polycarbonate or cellulose ester, and the polyamide is selected from nylon-6 or nylon-6,6.

15. The method according to claim 7, wherein at the same time, migration characteristics in the plastic, determinable according to DIN 53775-3, are reduced.

16. The method according to claim 7, wherein at the same time, the thermal stability of dyes in the plastic, determinable according to DIN EN 12877, is increased.

17. A process for the production of sulfonyl-bridged perinones of the formula (I) or isomeric forms thereof 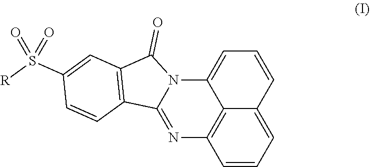
wherein
R is C₁-C₆-alkyl, halogen, nitro, aryl, aryloxysulfonyl, hydroxy, C₁-C₆-alkoxy, aryloxy, optionally alkyl- or acyl-substituted amino, optionally alkyl- or aryl-substituted aminosulfonyl, or a fused-on cycloaliphatic or heterocyclic radical,
comprising reacting sulfonyl-substituted isobenzofurandiones with at least one aromatic diamine from the group of naphthylene-1,8-diamine, chloronaphthylene-1,8-diamine, dichloronaphthylene-1,8-diamine, methylnaphthylene-1,8-diamine, dimethylnaphthylene-1,8-diamine, methoxynaphthylene-1,8-diamine, ethoxynaphthylene-1,8-diamine, acetaminonaphthylene-1,8-diamine and 1,8-diaminoacenaphthylene.

18. An extruded, blow-moulded or injection-moulded product comprising at least one sulfonyl-bridged perinone of the general formula (I) or isomers thereof 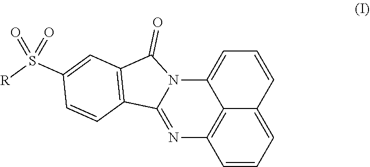
wherein
R is C₁-C₆-alkyl, halogen, nitro, aryl, aryloxysulfonyl, hydroxy, C₁-C₆-alkoxy, aryloxy, optionally alkyl- or acyl-substituted amino, optionally alkyl- or aryl-substituted aminosulfonyl, or a fused-on cycloaliphatic or heterocyclic radical, and
at least one plastic.

19. The extruded, blow-moulded or injection-moulded product according to claim 18, wherein the plastic is at least one thermoplastic.

20. The extruded, blow-moulded or injection-moulded product according to claim 18, wherein the at least one plastic is selected from the group consisting of vinyl polymers, polyesters, polyolefins and polyamides.
